(19) United States
(12) Patent Application Publication
(10) Pub. No.: US 2022/0296402 A1
(43) Pub. Date: Sep. 22, 2022

(54) WEIGHTED ANKLE FOOT ORTHOSIS

(71) Applicants: The Johns Hopkins University, Baltimore, MD (US); Ability Prosthetics & Orthotics, Inc., Exton, PA (US)

(72) Inventors: Marlies Eleana BEERLI CABELL, Frederick, MD (US); Temrah Miller OKONSKI, Ellicott City, MD (US)

(21) Appl. No.: 16/642,738

(22) PCT Filed: Aug. 31, 2018
(86) PCT No.: PCT/US2018/049124

§ 371 Date: Feb. 27, 2020

Related U.S. Application Data
(60) Provisional application No. 62/553,626, filed on Sep. 1, 2017.

(51) Int. Cl. A61F 5/01; A63B 21/065; A63B 21/00

(52) U.S. Cl. CPC A61F 5/0113; A63B 21/065; A63B 21/4011

(57) ABSTRACT

in some implementations, an ankle foot orthosis (AFO) may include a brace configured to support an ankle and a foot. The brace may include a foot portion to house the foot and a leg portion, extending upward from the foot portion, to house a leg. The AFO may include a weight compartment formed on or affixed to the leg portion of the brace, wherein the weight compartment is configured to house one or more weighted objects. In some implementations, an AFO attachment may include a plastic material formed into a curve with a limited range of flexibility, wherein the plastic material is configured to be attached to an AFO. The AFO attachment may include a weight compartment formed on or affixed to the plastic material, wherein the weight compartment is configured to house one or more weighted objects. Methods for making an AFO and AFO attachment are also disclosed.

### RELATED APPLICATION

This application claims priority to U.S. Provisional Patent Application No. 62/553,626, filed on Sep. 1, 2017, which is expressly incorporated by reference herein.

### BACKGROUND

An ankle foot orthosis (AFO) is a support intended to control the position and/or motion of the ankle, compensate for weakness, or correct deformities. An AFO can be used to support weak limbs or to position a limb into a more normal position. Furthermore, an AFO may be used to control foot drop caused by a variety of neurologic and musculoskeletal disorders. An AM can be used to stabilize the foot and ankle and provide toe clearance during the swing phase of gait. This helps decrease the risk of catching the toe and falling. A typical AFO creates an L-shaped frame around the foot and ankle, extending from just below the knee to the metatarsal beads of the foot.

### SUMMARY

According to some possible implementations, an ankle foot orthosis (AFO) may include a brace configured to support an ankle and a foot. The brace may include a foot portion configured to house the foot, and a leg portion, extending upward from the foot portion, configured to house a leg. The AFO may include a weight compartment formed on or affixed to the leg portion of the brace. The weight compartment may be configured to house one or more weighted objects.

According to some possible implementations, an AFO attachment may include a plastic material formed into a curve with a limited mime of flexibility. The plastic material may be configured to be attached to an. AFO. The AFO attachment may include a weight compartment formed on or affixed to the plastic material. The weight compartment may be configured to house one or more weighted objects.

According to some possible implementations, a method of making an AFO may include placing a material, for forming a weight compartment on a portion of the AFO, on a base material formed for the ankle foot orthosis. The method may include vacuum forming plastic around the material so as to affix the material to the base material.

### BRIEF DESCRIPTION OF THE DRAWINGS

FIG. 1 is a diagram of an example weighted ankle foot orthosis described herein.

FIG. 2 is a diagram of another example weighted ankle foot orthosis described herein.

FIG. 3 is a diagram of another example weighted ankle foot orthosis described herein.

FIG. 4 is a diagram of another example weighted ankle foot orthosis described herein.

FIG. 5 is a diagram of an example ankle foot orthosis attachment described herein.

FIG. 6 is a diagram of an example weighted ankle foot orthosis kit described herein.

FIG. 7 is a flow chart of an example process for making a weighted ankle foot orthosis described herein.

### DETAILED DESCRIPTION

The following detailed description of example implementations refers to the accompanying drawings. The same reference numbers in different drawings may identify the same or similar elements.

An ankle foot orthosis (AFO) may be used to control the position and/or motion of an ankle, compensate for weakness in the leg, foot, and/or ankle, correct deformities, support weak limbs, position a limb into a more normal position, control foot drop, stabilize the foot and ankle, provide toe clearance during the swing phase of gait, and/or the like. An AFO may be formed as an L-shaped frame around the foot and ankle, and may extend from below the knee (or above the knee, in sonic cases) to the metatarsal heads of the foot, or possibly beyond the metatarsal heads of the foot (although other heights, sizes, shapes, and configurations are possible).

Over time, as material science and material fabrication techniques have advanced, ankle foot orthoses have become lighter and less restrictive while still being resistant to breaking, Although a lightweight AFO may provide various benefits to a wearer, such as comfort, more natural movement, less restriction in movement, and/or the like, some wearers would benefit from a heavier AFO. For example, proprioceptive feedback provided by the nerves in the toes, foot, ankle, leg, and/or hip may be improved by increasing and/or customizing the weight of the AFO, such as in wearers affected by cerebral palsy (e.g., dystonic cerebral palsy, hemiplegic cerebral palsy, and/or the like), toe walking (e.g., due to neurological high tone, neurological low tone, premature birth, sensory defensiveness, autism, and/or the like), a stroke, a spinal cord injury, involuntary movements, impaired proprioception, and/or the like, Proprioception refers to the ability to sense the relative position of one's own body parts (e.g., awareness of body position in space), and proprioceptive feedback may be sensed in the brain based on proprioceptive feedback from nerves in the body. The AFO described herein may improve proprioceptive feedback provided to the brain, thereby improving gait, body movement, and/or the like. Additional details are described below.

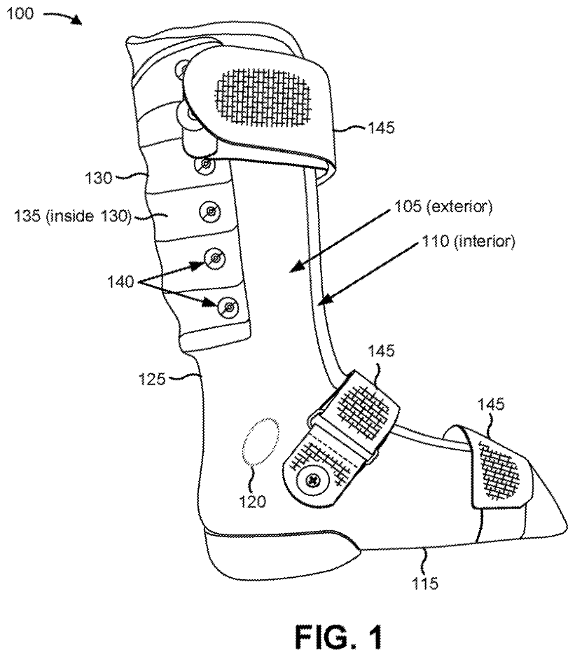

FIG. 1 is a diagram of an example weighted ankle foot orthosis (AFO) 100. As shown in FIG. 1, AFO 100 may include a brace 105 configured (e.g., formed, molded, fabricated, manufactured, and/or the like) to support an ankle and a foot. of a wearer. In some implementations, the brace 105 may be formed from a plastic material, a thermoplastic material, and/or the like. Additionally, or alternatively, the brace 105 may be formed from a carbon fiber material. The brace 105 may partially encase a foot of a wearer, an ankle of the wearer, and at least a portion of a leg of the wearer (e.g., above the ankle and below the knee, or above the ankle and extending above the knee). In some implementations, the brace may contain and/or be attached to (e.g., molded to, formed on, affixed to, and/or the like) an exterior portion of an interior lining 110, such as an interior lining 110 formed from a plaster cast formed from the wearer's body (e.g., foot, ankle, and leg) A soft material (e.g., cotton and/or the like) may be attached to the interior lining 110 for comfort of the wearer.

As shown, the brace 105 may include a foot portion 115, an ankle portion 120, and a leg portion 125. The foot. portion 115 may be configured to house (e.g., support, partially encase, and/or the like) the foot of the wearer. In some implementations, an anterior side and/or a proximal side of the foot portion 115 may be open (e.g., may not encase the foot). The ankle portion 120 may be configured to house (e.g., support, partially encase, and/or the like) the ankle of the wearer, In some implementations, an anterior side of the ankle portion 120 may be open (e.g., may not encase the ankle). The leg portion 125 may be configured to house (e.g., support, partially encase, and/or the like) the leg of the wearer. In some implementations, an anterior side and/or a proximal side of the leg portion 125 may be open (e.g., may not encase the leg).

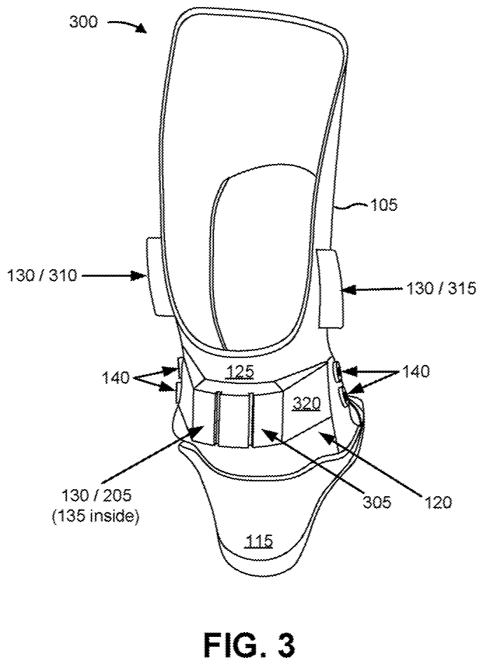

As shown, the leg portion 125 may extend upward from the foot portion 115, and the leg portion 125 and the foot portion 115 may meet at the ankle portion 120. As shown in FIG. 1, in some implementations, the brace 105 is non-articulable, meaning that the leg portion 125 and the foot portion 115 do not articulate with respect to one another (e.g., are substantially positionally fixed with respect to one another). In this case, the foot portion 115, the ankle portion 120, and the leg portion 125 may be fabricated simultaneously (e.g., using the same and/or a single vacuum forming process), and may all be formed from the same piece of material. In some implementations, the brace 105 is articulable, meaning that the leg portion 125 and the foot portion 115 articulate with respect to one another, as described in more detail below in connection with FIG. 3.

As further shown in FIG. 1, the AFO 100 may include a weight compartment 130 formed on and/or affixed to the leg portion 125 of the brace 105. The weight compartment 130 may be configured to house (e.g., contain, hold, encase, partially encase, and/or the like) one or more weighted objects 135. In some implementations, such as the AFO 100 of FIG. 1, the weight compartment 130 may not be exposed (e.g., to the air, to an exterior of the brace 105, and/or the like), and may be fully encased by the brace 105, an interior lining, 110 attached to the brace 105, or both. In this case, the weighted object 135 may be fully encased within the weight compartment 130. which may be formed by a vacuum process (e.g., vacuum molding, vacuum forming, vacuum sealing, and/or the like) to attach a plastic material to a base material (e.g., used to form the interior lining 110), to form the brace 105. The weight compartment 130 may seal the weighted object 135 tightly between the brace 105 and the interior lining 110 (e.g., using the vacuum process), so as to reduce or prevent movement of the weighted object 135, thereby improving proprioceptive feedback by reducing or eliminating the sensing of movement that is separate from body movement (e.g., movement of the weighted object 135 that is not synchronized with movement of the body). As used herein, vacuum molding, vacuum forming, and vacuum sealing may be used interchangeably to refer to substantially the same vacuum process.

In the fabrication process, a cast of the wearer's limb (e.g., leg, ankle, and/or foot) may be filled with plaster of Paris or a similar material to form a positive three dimensional shape of the limb. A base material, for forming the interior lining 110, may be placed on the positive three dimensional shape, and the weight compartment 130 may then be formed on the base material of the interior lining 110, as described elsewhere herein

In some implementations, the weighted object 135 may include a metal. For example, the weighted object 135 may include a non-lead metal, such as tungsten, steel (e.g., stainless steel and/or the like), iron (e.g., cast iron and/or the like), brass, bismuth, and/or the like. In the example AFO 100 of FIG. 1, the weighted object 135 may be a material, such as sheet metal, placed on (e.g., folded around, formed around, bent around, and/or the like) and/or attached to the interior lining 110 (e.g., a posterior portion of the interior lining 110), and vacuum sealed between the interior lining 110 and the brace 105 by vacuum forming a plastic around the sheet metal to seal the sheet metal between the interior lining 110 and the brace 105. In this case, the weighted object 135 may be completely sealed between the plastic (e.g., that forms the brace 105) and the interior lining 110 so that the weight compartment 130 and/or the weighted object 135 is not removable from the weight compartment 130 (e.g., except by ripping through and/or destroying the brace 105 and/or the interior lining 110). As will be described in more detail below, other forms of weighted objects 135 may be used.

In some implementations, the total amount of weight of the weighted ° bleats) 135 added to an AFO (e.g., AFO 100 or one or more other AFOs described herein) may be greater than zero pounds and less than 100 pounds. For example, the total amount of weight of the weighted object(s) 135 added to the AFO may be greater than zero pounds, less than 100 pounds, between zero and 20 pounds, between 0.25 and 5 pounds, up to 25 pounds, and/or the like. In some implementations, where multiple weighted objects 135 are used, the total amount of weight added to the MO can vary by an increment of weight of a single weighted object 135. For example, a single weighted object 135 may weigh 0.1 pounds, 0.25 pounds, 0.5 pounds, 1 pound, 2 pounds, and/or the like. In some implementations, different weighted objects 135 may have different weights. In some implementations, different weighted objects 135 may have the same weight. Where the terms “pound” or “pounds” are used herein, another unit of weight measure may replace those terms, such as “kilogram” or “kilograms.” in some implementations, the amount of weight added to the AFO may depend on one or more factors, such as the weight of the wearer of the AFO, the strength of the wearer, an ailment affecting the wearer, a weight of the AFO, a type of the AFO, a design of the AFO, a material of the AFO, and/or the like.

As further shown in FIG. 1, in some implementations, the weight compartment 130 and/or the weighted object 135 may be further secured to the AFO 100 using one or more fasteners 140. In FIG. 1, the fasteners 140 are shown as rivets, but other fasteners 140 may be used, such as screws, bolts, and/or the like, as described in more detail below. When the weighted object 135 is affixed to the AFO 100 using vacuum sealing, such fasteners 140 may optionally be used to further reduce or eliminate movement of the weighted object 135 (e.g., within the weight compartment 130 and/or the AFO 100), thereby further improving proprioceptive feedback. FIG. 1 further shows multiple straps 145. In some implementations, a strap 145 may further secure the weight compartment 130 and/or the weighted object 135 to the AFO 100. Additionally, or alternatively, the straps 145 may be used to hold the foot, ankle, and/or leg of the wearer in place in the AFO 100, and/or to secure the foot, ankle, and/or leg of the wearer in the AFO 100. Thus, detaching the strap 145 may not impact a degree of movement of the weighted object 135 (e.g., within the weight compartment 130 and/or the AFO 100), in some implementations.

In FIG. 1, the weight compartment 130 is shown as being formed on and/or affixed to the leg portion 125 of the brace 105 above the ankle portion 120 of the brace 105. In some implementations, the weight compartment 130 may be formed on and/or affixed to the leg portion 125 at the ankle portion 120 (e.g., may be formed on and/or affixed to the ankle portion 120) or below the ankle portion 120. Additionally, or alternatively, the weight compartment 130 may be formed on and/or affixed to the foot portion 115 of the brace 105. Furthermore, although the weight compartment 130 of FIG. 1 is shown as being formed on and/or affixed to a posterior side of the brace 105, in some implementations, the weight compartment 130 may be formed on and/or affixed to an anterior side of the brace 105, one or more lateral sides of the brace 105, and/or the like (e.g., for different types and/or designs of AFOs). The weight compartment 130 and/or the weighted object 135 max be positioned on the AFO 100 and/or the brace 105 depending on the needs of the wearer. In some implementations, a single weight compartment 130 may be formed on and/or affixed to the brace 105. In some implementations, multiple weight compartments 130 may be formed on and/or affixed to the brace 105,

In some implementations, the weight compartment 130 of the AFO 100 may be non-tunable, meaning that a weight of the weight compartment 130 cannot be modified after the weight compartment is formed on and/or affixed to the brace 105. Furthermore, the weight compartment 130 of the AFO 100 may be non-removable, meaning that the weight compartment 130 may not be removed from the AFO 100 (e.g., except by ripping through and/or destroying the brace 105 and/or the interior lining 110). Thus, the AFO 100 may be referred to as a non-tunable, non-removable weighted AFO 100, or the weight compartment 130 of the AFO 100 may be referred to as a non-tunable, non-removable weight compartment 130.

As indicated above, FIG. 1 is provided as an example, Other examples are possible and may differ from what was described with regard to FIG. 1.

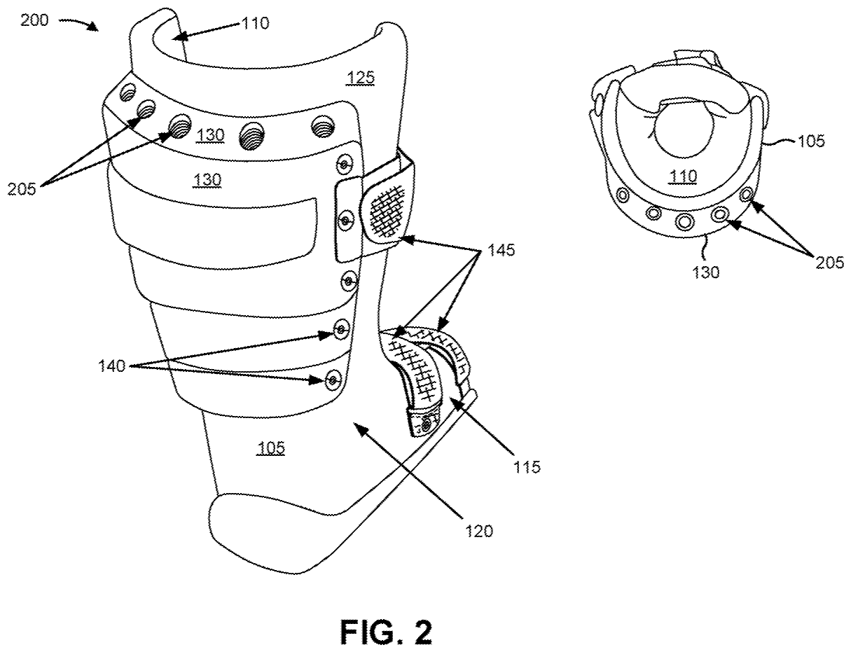

FIG. 2 is a diagram of another example weighted ankle foot orthosis 200. As shown in FIG. 2, the AFO 200 may include a brace 105, an interior lining 110, a foot portion 115, an ankle portion 120, a leg portion 125, a weight compartment 130 configured to house one or more weighted objects 135, one or more fasteners 140, and/or one or more straps 145, as described above in connection with FIG. 1. The weight compartment 130 of the AFO 200 may be tunable, meaning that a weight of the weight compartment 130 may be modified after the weight compartment is formed on and/or affixed to the brace 105 (e.g., by adding or removing one or more weighted objects 135 to or from the weight compartment 130), Furthermore, the weight compartment 130 of the AFO 200 may be non-removable, meaning that the weight compartment 130 may not be removed from the AFO 200 (e.g., except by ripping through and/or destroying the brace 105 and/or the interior lining 110). Thus, the AFO 200 may be referred to as a tunable, non-removable weighted AFO 200, and/or the weight compartment 130 of the AFO 200 may be referred to as a tunable, non-removable weight compartment 130.

As further shown in FIG. 2, in some implementations, the weight compartment 130 may be exposed (e g., to the air, to an exterior of the brace 105, and/or the like), and may be partially encased by the brace 105, an interior lining 110 attached to the brace 105, or both. In this case, one or more weighted objects 135 may be partially encased within the weight compartment 130, which may include one or more cavities 205 for housing (e.g., holding, securing, partially encasing, and/or the like) the one or more weighted objects 135. In some implementations, a cavity 205 may be configured (e.g., formed, molded, drilled, created by removing material, and/or the like) to restrict or substantially prevent movement of a weighted object 135 when the weighted object 135 is housed in the cavity 205.

For example, a cavity 205 may be formed to be the same size and/or shape as a single weighted object 135 to be housed in the cavity 205 (or a slightly larger size to permit the singled weighted object 135 to be inserted into and/or removed from the cavity 205). Additionally, or alternatively, a cavity 205 may be formed to be the same size and/or shape as a group of weighted objects 135 to be housed in the cavity 205 (or a slightly larger size to permit the one or more weighted objects 135 of the group of weighted objects 135 to be inserted into and/or removed from the cavity 205). Additionally, or alternatively, the walls of the cavity 205 may be formed from and/or made of a material with a relatively high coefficient of friction, such as rubber, foam, and/or the like. Additionally, or alternatively, one or more magnets may be formed and/or included in the cavity 205 (e.g., to restrict movement when the weighted object 135 is metal). In this way, movement of the weighted object 135 may be restricted, thereby enhancing proprioceptive feedback.

In some implementations, a cavity 205 may be formed by a vacuum process (e.g., vacuum molding, vacuum forming, vacuum sealing, and/or the like) to attach a plastic material to a base material (e.g., used to form the interior lining 110), to form the brace 105. For example, a cavity 205 of a weight compartment 130 may be formed by placing and/or attaching a material (e.g., a plastic material, a metal material, a magnetic material, a foam material, a rubber material, and/or the like) to the base material (e.g., used to form the interior lining 110) and/or the leg portion 125 of the brace 105, and by vacuum sealing the material between the base material and the brace 105. In some implementations, the material may include a cavity (e.g., a hollow tube, in the example of FIG. 2), and such vacuum sealing may form the cavity 205 in the weight compartment 130. Additionally, or alternatively, at least a portion of the material (e.g., some or all of the material) may be removed from the weight compartment 130, after the vacuum sealing process, to form the cavity 205. In this case, the vacuum sealing may partially seal the material between the brace 105 and the interior lining 110 (e.g., leaving an opening of cavity 205), which may permit one or more weighted objects 135 to be inserted into and/or removed from the weight compartment 130 (e.g., via one or more cavities 205) after the AFO 200 is formed (e.g., after the vacuum sealing process is complete).

As shown in FIG. 2, in some implementations, the AFO 200 may include multiple cavities 205. One or more of the multiple cavities 205 may be configured to permit insertion or removal of a weighted object 135. For the example AFO 200 of FIG. 2, the weighted object 135 may be a rod (e.g., metal rod and/or the like). and the cavities 205 may be rod-shaped (e.g., cylindrical). However, other shapes of the weighted object 135 and/or the cavities 205 are possible.

By including multiple cavities 205 in the weight compartment 130, the AFO 200 may be modified as the wearer grows, as the wearer's condition changes, as the wearer becomes stronger or weaker, and/or the like. In some cases, insurance of the wearer may cover a limited number of AFOs per time period (e.g., two per year, one per year, one every five years, and/or the like). Furthermore, new AFOs may be expensive to produce. Thus, by creating an AFO 200 with a tunable weight compartment 130 configured to hold different numbers of weighted objects 135 (e.g., anywhere from zero to five weighted objects 135 in the example AFO 200), expenses may be reduced by increasing the life span of a single AFO as compared to replacing the AFO multiple times as the wearer changes.

As indicated above, FIG. 2 is provided as an example. Other examples are possible and may differ from what was described with regard to FIG. 2.

FIG. 3 is a diagram of another example weighted ankle foot orthosis 300, As shown in FIG. 3, the AFO 300 may include a brace 105 (but may not include a separate interior lining 110, in some cases), a foot portion 115, an ankle portion 120, a leg portion 125, a weight compartment 130 configured to house one or more weighted objects 135, one or more fasteners 140, and/or one or more straps 145 (not shown, and which may or may not be included in the AFO 300), as described above in connection with FIGS. 1 and 2. The weight compartment 130 of the AFO 300 may be tunable and removable. For example, the weight compartment 130 may be affixed to and removable from the brace 105. Thus, the AFC 300 may be referred to as a tunable, removable weighted AFO 300, and/or the weight compartment 130 of the AFO 300 may be referred to as a tunable, removable weight compartment 130. Additionally, or alternatively, the weighted AFO 300 may be an articulated AFO 300 because the leg portion 12.5 and the foot portion 115 are configured to articulate with respect to one another (e.g., at or near an ankle portion 120).

As shown in FIG. 3, the fasteners 140 of the AFO 300 may permit the weight compartment 130 to be removed from the AFO 300 (e.g., the brace 105). For example, a fastener 140 may include a screw fastener, such as a Chicago screw (also known as a sex bolt, a barrel nut, a barrel bolt, a post and screw, a connector bolt, and/or the like), as shown in FIG. 3. FIG. 3 shows four Chicago screws, two on each lateral side of the brace 105. However, a different type of fastener 140 may be used to affix the weight compartment 130 to the AFO 300, such as a snap fastener, a keyhole fastener, and/or the like. Additionally, or alternatively, a different number of fasteners 140 may be used to affix the weight compartment 130 to the AFO 300.

As shown in FIG. 3, in some implementations, the AFO 300 may include multiple weight compartments, and/or may include weight compartments in different positions than those of FIGS. 1 and 2. For example, a first weight compartment 305 is shown on a posterior side of the leg portion 125 of the brace 105, closer to the ankle portion 120 than the weight compartment 130 of FIGS. 1 and 2. Furthermore, a second weight compartment 310 and a third weight compartment 315 are shown on opposite lateral sides (e.g., a lateral side and a medial side) of the leg portion 125 of the brace 105.

The weight compartments 130 shown in FIG. 3 may each include one or more cavities 205 for housing one or more weighted objects 135, which are shown as being rectangular in shape. In some aspects, these weight compartments 130 may be tunable, such that one or more weighted objects 135 may be added to or removed from the weight compartments 130 via the one or more cavities 205. Additionally, or alternatively, the AFO 300 may be tunable by removing the weight compartment 130 (e.g., by unfastening fasteners 140) and attaching a different weight compartment 130 that houses weighted objects 135 having a different weight.

In some implementations, the weight compartment 130 may be referred to as and/or included in an AFO attachment 320. The AFO attachment 320 may be formed from a material (e.g., a plastic material, a carbon fiber material, and/or the like) formed into a curve with a limited range of flexibility (e.g., that prevents the curve from being completely flattened into a plane). The AFO attachment 320 may include and/or may be configured to receive one or more fasteners .140, such that the AFO attachment 320 is affixable to the AFO 300, The weight compartment 130 may be formed on and/or affixed to the material of the AFO attachment 320 (e.g., using vacuum forming, one or more fasteners 140, and/or the like). In this way, the weight compartment 130 may be removable from the AFO 300, thereby permitting the amount of weight to be adjusted. as the wearer changes, reducing cost, increasing the life span of the AFO 300, and/or the like.

As indicated above, FIG. 3 is provided as an example, Other examples are possible and may differ from what was described with regard to FIG. 3.

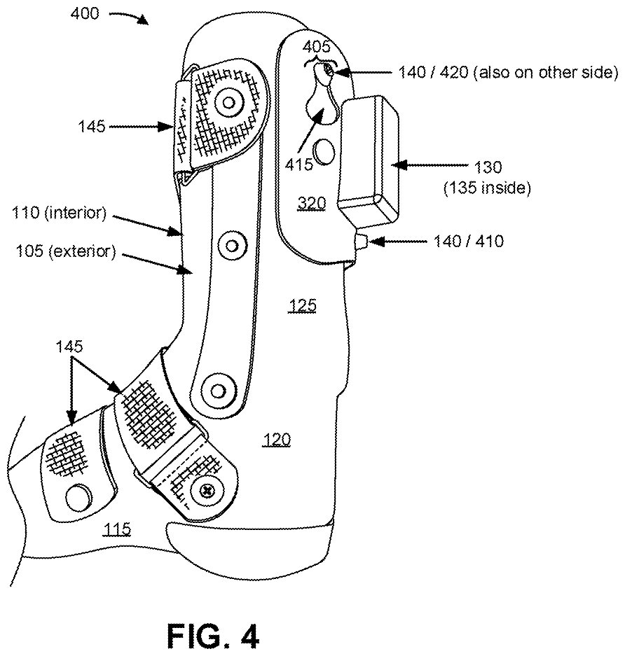

FIG. 4 is a diagram of another example weighted ankle foot orthosis 400. As shown in FIG. 4, the AFO 400 may include a brace 105, an interior lining 110, a foot portion 115, an ankle portion 120, a leg portion 125, a weight compartment 130 configured to house one or more weighted objects 135, one or more fasteners 140, and/or one or more straps 145, as described above in connection with FIGS. 1-3. The weight compartment 130 of the AFO 400 mays be affixed to a removable AFO attachment 320, thus making the weight compartment 130 removable and the AFO 400 tunable. For example, the weight compartment 130 may be affixed to and removable from the brace 105 (e.g., a leg portion 125 of the brace 105) via the AFO attachment 320. In some implementations, the brace 105 of AFO 400 is non-articulable. In some implementations, the brace 105 of AFO 400 is articulated.

As shown in FIG. 4, the fasteners 140 of the AFO 400 may permit the weight compartment 130 to be removed from the AFO 400 (e.g., the brace 105). For example, in FIG. 4, the weight compartment 130 is affixed to an AFO attachment 320, which is affixed to the brace 105 using two keyhole fasteners 405 and a snap fastener 410. As shown, the AFO attachment 320 may include a keyhole 415 formed on the AFO attachment 320. In this case, the AFO attachment 320 may be attached to the AFO 400 by inserting a keyhole post 420, affixed to the brace 105 (e.g., a leg portion 125 of the brace 105) into a wide portion of the keyhole 415 and sliding the AFO attachment 320 down to secure the keyhole post 420 in a narrow portion of the keyhole 415.

Additionally, or alternatively, the AFO attachment 320 may include a first unmated portion (e.g. a male portion) of a snap fastener 410 affixed to the AFO attachment 320, and the AFO 400 may include a second unmated portion (e.g. a female portion) of a snap fastener 410 affixed o the brace 105. The AFO attachment 320 may be affixed to the brace 105 may securing the keyhole post 420 in the narrow portion of the keyhole 415 and connecting (e.g., inserting, snapping, and/or the like) the first unmated portion and the second unmated portion of the snap fastener 410. In this way, the AFO attachment 320 may be easily attached to and removed from the AFO 400 while reducing or preventing movement of weighted object(s) 135 supported by the AFO attachment 320. As described elsewhere herein, the AFO attachment 320 may be affixed to the brace 105 using different types and/or numbers of fasteners 140 than shown in FIG. 4.

In some implementations, the weight compartment 130 may include one or more cavities 205 to permit one or more weighted objects to be inserted into and/or removed from the weight compartment 130. Additionally, or alternatively, the weight compartment 130 may be affixed to an AFO attachment 320, and the AFO attachment 320 may be removable from the brace 105 to permit attachment of a different AFO attachment 320 with a different amount of weight. Additionally, or alternatively, the weight compartment 130 may be removable from the AFO attachment 320 to permit a different weight compartment 130. with a different amount of weight, to be attached to the AFO attachment 320. One or more of these techniques may be used to make the AFO 400 tunable, thereby permitting the amount of weight to be adjusted as the wearer changes, reducing cost, increasing the life span of the AFO 400, and/or the like.

As indicated above, FIG. 4 is provided as an example. Other examples are possible and may differ from what was described with regard to FIG. 4.

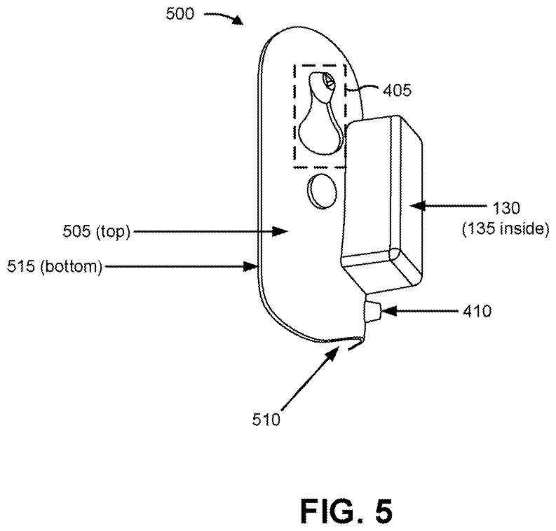

FIG. 5 is a diagram of an example ankle foot orthosis attachment 500. As shown in FIG. 5, the AFO attachment 500 may include a weight compartment 130 configured to house one or more weighted objects 135, as described above in connection with FIGS. 1-4. In some implementations, the weight compartment 130 may not be removable from the AFO attachment 500 (e.g., except by ripping through and/or destroying the material of the AFO attachment 500). In some implementations, the weight compartment 130 may be removable from the AFO attachment 500 (e.g., via one or more fasteners 140).

As further shown in FIG. 5, the AFO attachment 500 may include a material 505 (e.g., a plastic material) formed into a curve 510. The curve 510 may have a limited range of flexibility (e.g., 5 degrees, 10 degrees, 15 degrees, 20 degrees, and/or the like) to be pliable enough to permit the AFO attachment 500 to be attached to AFOs of different sizes while also being of sufficient strength to resist damage and/or prevent or reduce movement of the weighted objects 135, In some implementations, the limited range of flexibility prevents the curve 510 of the AFO attachment 500 from being completely flattened

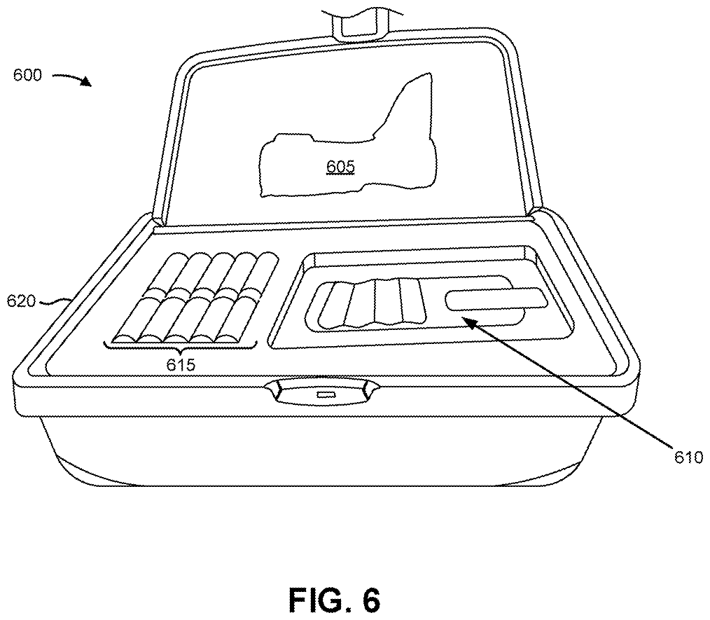

The material 505 may be configured (e.g., formed) to be attached to an AFO. For example, the AFO attachment 500 may include at least one fastener 140 for attaching the AFO attachment 500 (e.g., via the material 505) to an AFO. As described above in connection with FIG. 4, the fastener(s) 140 max include any number of keyhole fasteners 405, snap fasteners 410, and/or the like. Additionally, or alternatively, the fastener(s) 140 may include any number of screw fasteners. In some implementations, the fastener 140 may include any number of hook-and-loop fasteners (e.g., a Velcro fastener and/or the like), such as when the AFO attachment 500 is designed for an AFO kit 600, as described below in connection with FIG. 6,

As further shown, the AFO attachment 500 may include a weight compartment 130 formed on and/or affixed to the material 505. As described elsewhere herein, the weight compartment 130 may be configured to house one or more weighted objects 135 (e.g., in a tunable or non-tunable fashion). In some implementations, the weight compartment 130 may be formed by a vacuum process to attach a plastic material (e.g., material 505) to a base material 515 (e.g., a pliable plastic material and/or the like) to form the AFO attachment 500. The weight compartment 130 may seal the weighted object 135 tightly between the material 505 and the base material 515 (e.g., using the vacuum process), so as to reduce or prevent movement of the weighted object 135, thereby improving proprioceptive feedback. In this case, the weight compartment 130 may not be exposed, and may be fully encased by the material 505, the base material 515, or both. Alternatively, the weight compartment 130 may be partially exposed and/or may be formed with one or more cavities 205, using a similar process as described above in connection with FIG. 2.

As indicated above, FIG. 5 is provided as an example. Other examples are possible and may differ from what was described with regard to FIG. 5.

FIG. 6 is a diagram of an example weighted ankle foot orthosis kit 600. AFO kit 600 may be used by a practitioner to determine the appropriate amount of weight to be attached to an AFO for a wearer. As shown, the AFO kit 600 may include an AFO 605 (e.g., having a specific site, having an adjustable size, one or more of the AFOs described elsewhere herein, and/or the like). Additionally, or alternatively, the AFO kit 600 may include an AFO attachment 610, such as the AFO attachment 320, the AFO attachment 500, and/or the like, described elsewhere herein. In some implementations, the AFO attachment 610 included in the AFO kit 600 may be tunable (e g., may include one or more cavities 205) so that the practitioner can test different amounts of weight for a wearer. Additionally, or alternatively, the AFO kit 600 may include one or more weighted objects 615, such as the weighted object(s) 135 described elsewhere herein. The weighted objects 615 may include multiple weighted objects 615 having the same weight, and/or may include multiple weighted objects 615 having different weights. In some implementations, the AFO kit 600 max include a case 620 (e.g., a housing and/or the like) fitted with compartments for holding the AFO 605, the AFO attachment 610, and/or the weighted objects 615. In this way, a practitioner can test different amounts of weight for a wearer before obtaining a custom AFO for the wearer, thereby reducing cost.

As indicated above, FIG. 6 is provided as an example. Other examples are possible and may differ from what was described with regard to FIG. 6.

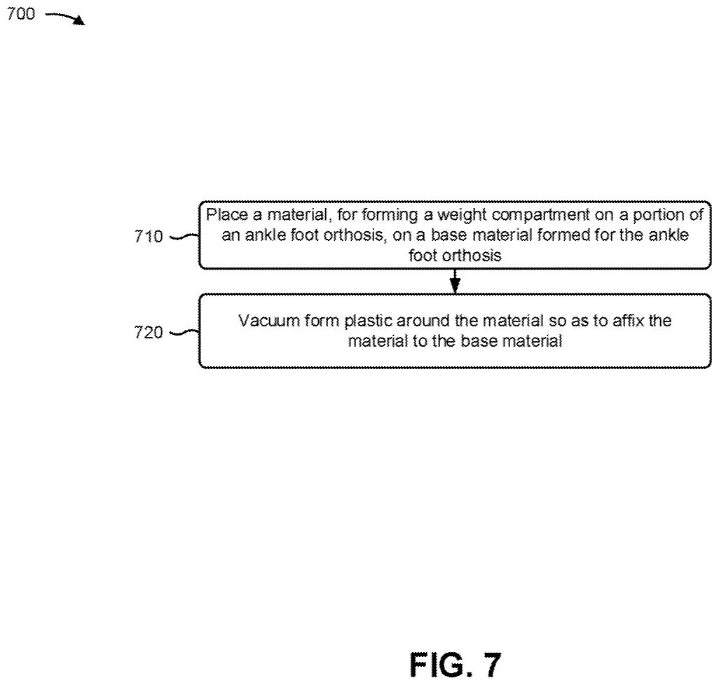

FIG. 7 is a flow chart of an example process 700 for making a weighted ankle foot orthosis. Process 700 may be performed, for example, by a vacuum forming machine, a vacuum forming system, and/or the like,

As shown in FIG. 7, process 700 may include placing a material, for forming a weight compartment on a portion of an ankle foot orthosis, on a base material formed for the ankle foot orthosis (block 710). For example, a material for forming a weight compartment on a portion of an AFO and/or an AFO attachment may be placed (e.g., by a machine, by a machine operator, and/or the like) on an interior lining and/or a base material (e.g. a base material from which the interior lining is formed) formed for the AFO and/or the AFO attachment, as described in more detail above in connection with one of more of FIGS. 1-6.

As further shown in FIG. 7, process 700 may include vacuum forming plastic around the material so as to affix the material to the base material (block 720), For example, plastic may be vacuum formed around the material (e.g., by a vacuum forming machine, a vacuum forming system, and/or the like) so as to affix the material to the interior lining and: or the base material, as described in more detail above in connection with one of more of FIGS. 1-6.

Process 700 may include additional implementations, such as any single implementation or any combination of implementations described below and/or in connection with one or more other processes described elsewhere herein.

In some implementations, the vacuum forming completely seals the material between the plastic and the interior lining (or the base material) so that the material is not removable from the weight compartment. In some implementations, the vacuum forming partially seals the material between the plastic and the interior lining (or the base material) to permit insertion or removal of one or more weighted objects in the weight compartment formed by partially sealing the material between the plastic and the interior lining (or the base material). In some implementations, process 700 may include removing at least a portion of the material from the vacuum formed plastic to form the weight compartment.

Although FIG. 7 shows example blocks of process 700, in some implementations, process 700 may include additional blocks, fewer blocks, different blocks, or differently arranged blocks than those depicted in FIG. 7. Additionally, or alternatively, two or more of the blocks of process 700 may be performed in parallel.

The foregoing disclosure provides illustration and description, but is not intended to be exhaustive or to limit the implementations to the precise form disclosed. Modifications and variations are possible in light of the above disclosure or may be acquired from practice of the implementations.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an' are intended to include one or more items, and may be used interchangeably with one or more.” Furthermore, as used herein, the term “set' is intended to include one or more items (e.g., related items, unrelated items, a combination of related and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. An ankle foot orthosis, comprising;
a brace configured to support an ankle and a foot, wherein the brace includes:
a foot portion configured to house the foot, and
a leg portion, extending upward from the foot portion, configured to house a leg; and
a weight compartment formed on or affixed to the leg portion of the brace, wherein the weight compartment is configured to house one or more weighted objects.

2. The ankle foot orthosis of claim 1, wherein the weight compartment is vacuum formed on the leg portion of the brace.

3. The ankle foot orthosis of claim 1, wherein the one or more weighted objects are housed within the weight compartment as a result of the weight compartment being vacuum formed on the leg portion of the brace,

4. The ankle foot orthosis of claim 1, wherein the weight compartment is affixed to and removable from the leg portion of the brace.

5. The ankle foot orthosis of claim 1, wherein the weight compartment is affixed to the leg portion of the brace using at least one of:
one or more keyhole fasteners,
one or more snap fasteners,
one or more screw fasteners, or
one or more rivets.

6. The ankle foot orthosis of claim 1, wherein the weight compartment is formed on or affixed to the leg portion of the brace at or above an ankle portion of the brace configured to support the ankle.

7. The ankle foot orthosis of claim 1, wherein the weight compartment includes one or more cavities for housing the one or more weighted objects,
wherein the one or more cavities are configured to prevent movement of the one or more weighted objects.

8. The ankle foot orthosis of claim 1, wherein the weight compartment includes multiple cavities,
wherein each cavity, of the multiple cavities, is configured to permit insertion or removal of a weighted object of the one or more weighted objects.

9. The ankle foot orthosis of claim 1, wherein the weight compartment is positioned on a posterior side of the leg portion of the brace.

10. The ankle foot orthosis of claim 1, wherein the weight compartment is positioned on a lateral side or an anterior side of the leg portion of the brace.

11. The ankle foot orthosis of claim 1, wherein the leg portion is configured to articulate with respect to the foot portion.

12. An ankle foot orthosis attachment, comprising:
a plastic material formed into a curve with a limited range of flexibility, wherein the plastic material is configured to be attached to an ankle foot orthosis, and
a weight compartment formed on or affixed to the plastic material, wherein the weight compartment is configured to house one or more weighted objects.

13. The ankle foot orthosis attachment of claim 12, wherein the limited range of flexibility prevents the curve from being flattened.

14. The ankle foot orthosis attachment of claim 12, further comprising at least one fastener for attaching the ankle foot orthosis attachment to the ankle foot orthosis,

15. The ankle foot orthosis attachment of claim 14, wherein the fastener includes at least one of:
one or more hook-and-loop fasteners,
one or more keyhole fasteners,
one or more snap fasteners, or
one or more screw fasteners.

16. The ankle foot orthosis attachment of claim 12, Wherein the weight compartment includes one or more cavities that permit insertion and removal of the one or more weighted objects.

17. A method of making an ankle foot orthosis, comprising:
placing a material, for forming a weight compartment on a portion of the ankle foot orthosis, on a. base material formed for the ankle foot orthosis; and
vacuum forming plastic around the material so as to affix the material to the base material.

18. The method of claim 17, wherein the vacuum forming completely seals the material between the plastic and the base material so that the material is not removable from the weight compartment.

19. The method of claim 17, wherein the vacuum forming partially seals the material between the plastic and the base material to permit insertion or removal of one or more weighted objects in the weight compartment formed by partially sealing the material between the plastic and the base material.

20. The method of claim 17, further comprising removing at least a portion of the material from the vacuum formed plastic to form the weight compartment.
